(19) United States
(12) Patent Application Publication
(10) Pub. No.: US 2026/0209113 A1
(43) Pub. Date: Jul. 23, 2026

(54) HUMIDITY SENSOR PRODUCTION METHOD

(71) Applicant: Süleyman Demírel Üniversitesi Ídari Ve Mali Ísler Daíre Baskanligi Genel Sekreterlik, Isparta (TR)

(72) Inventors: Murat KALELI, ISPARTA (TR); Ibrahim SLAH, ISPARTA (TR)

(21) Appl. No.: 19/107,082

(22) PCT Filed: Nov. 15, 2024
(86) PCT No.: PCT/TR2024/051341

§ 371 Date: Feb. 27, 2025

(51) Int. Cl. C03C 17/06; G01N 27/22

(52) U.S. Cl. CPC C03C 17/06; G01N 27/227

(57) ABSTRACT

The present disclosure relates to the field of metal oxide semiconductor (MOS) sensors and relates to a humidity sensor (1) production method that enables the production of a NiOx-based humidity sensor (1) that is cost-effective, fast-responding, highly sensitive, has a wide relative humidity sensing range, stable, long-lasting, quickly recoverable, and can operate at room temperature and with low voltage by means of silver (Ag) interdigitated electrode contacts using the accumulation method.

### TECHNICAL FIELD

The present disclosure relates to the field of metal oxide semiconductor (MOS) sensors and relates to a humidity sensor production method that enables the production of a NiOx-based humidity sensor that is cost-effective, fast-responding, highly sensitive, has a wide relative humidity sensing range, stable, long-lasting, quickly recoverable, and can operate at room temperature and with low voltage by means of silver (Ag) interdigitated electrode contacts using the accumulation method.

### STATE OF THE ART

In today's industry, sensors are frequently used elements for many quantitative sensing processes such as level, temperature, distance, humidity, presence/absence. For these sensing processes, many technologies such as laser, ultrasonic, light, etc. are used to reach the quantitative value desired to be detected/measured. One of the most basic quantities that need to be measured/detected is humidity, and for this reason, various humidity sensors are developed.

While most humidity sensors are ceramic and metal oxide sensors, the biggest problems of these sensors are difficulty in production, low sensitivity, long response and recovery times (the best is 0.3 seconds), and negative hysteresis properties.

In the case of humidity sensors, the need for high-cost materials and/or material accumulation methods, the need for relatively high operating power (voltage 1-10 volts), the need to operate at high temperatures (room temperature—300 C), and the narrow/limited humidity detection ranges (humidity %—relative humidity range is best 5-98% relative humidity) can be listed as additional difficulties.

The document numbered CN112730528 A in the state of the art belongs to the relevant technical field of gas sensors and describes SnO2 doped NiO nanoparticles, a preparation method of an ethanol sensor and a product of an ethanol sensor with SnO2 doped NiO nanoparticles. The method comprises the process steps of S1: mixing a fuel with a co-solvent and then adding a tin-containing compound and a nickel-containing compound to the mixed solution to obtain a precursor solution, S2: performing ultrasonic treatment on the precursor solution to evenly mix the precursor solution, and S3: performing flame spray pyrolysis on the precursor solution evenly mixed in step S2 to form SnO2-doped NiO nanoparticles with a particle size of 5-50 nm. The invention also describes a method for in situ preparation and ex situ preparation of an ethanol sensor using SnO2 doped NiO nanoparticles and a product. According to the invention, the gas-sensitive property of the gas-sensitive material is improved and the applicability of the nanomaterial in the field of gas ethanol gas detection is increased.

In another prior art document numbered U.S. Pat. No. 10,670,547B2, a nano-structured nickel oxide environmental sensor device and a package encapsulating said device are disclosed. A nano-structured environmental sensor comprises a silicon-based substrate, a structural base positioned on the substrate, and a sensor portion suspended above the structural base. The upper surface of the sensor portion is formed from nickel oxide using atomic layer accumulation. The nano-structured thin film nickel oxide environmental sensor is provided in a housing to form an environmental sensor package for sensing the characteristics of the environment to which the environmental sensor package is exposed.

Application number KR100812357B1 describes an ultrasensitive metal oxide gas sensor and a method for manufacturing thereof. An ultrasensitive metal oxide gas sensor and a method for manufacturing thereof are provided for maximising gas diffusion by providing macro-pores between nanofibres. An ultrasensitive metal oxide gas sensor comprises a sensor electrode and a porous metal oxide thin film. The porous metal oxide thin film is formed on the sensor electrode and has a network structure consisting of nanofibres including nanorods formed by single crystals. The average width of the single crystals is 10 to 20 nm, and the average length is 50 to 100 nm. The porous metal oxide thin film has first pores between nanofibres and second pores between nanorods. The average size of the first pore is 55 to 70 nm, and the average size of the second pore is 10 to 25 nm.

In order to determine the state of the art, documents such as TR 2021/005440, TR 2016/02813, TR 2022/014113, TR 2019/00658, TR 2017/00087, CN113125520 B, RO134520 A2, EP3223702 B1 and EP3607109 B1 can also be examined.

As a result, developments are being made in humidity sensor production methods, therefore new applications are needed that will eliminate the disadvantages mentioned above and provide solutions to existing applications.

### THE AIM OF THE INVENTION

The present invention relates to a method of producing humidity sensors that meet the above-mentioned requirements, eliminate all disadvantages and provide some additional advantages.

The main aim of the invention is to provide a method of producing humidity sensors that enables the production of a NiOx-based humidity sensor that is cost-effective, fast-response, highly sensitive, has a wide relative humidity sensing range, stable, long-lasting, fast-recovery (0.4 seconds), and can operate at room temperature and with low voltage (millivolt and lower levels) by means of the silver (Ag) interdigitated electrode contacts on which the accumulation process is applied.

Another aim of the invention is to provide a method of producing an easy-to-process humidity sensor.

Another aim of the invention is to provide a method of producing a humidity sensor that enables the production of a humidity sensor with positive hysteresis properties.

Another aim of the invention is to provide a method of producing a humidity sensor that is cost-effective.

Another aim of the invention is to provide a method of producing a humidity sensor that enables the production of a humidity sensor with wide humidity/relative humidity sensing range.

Another aim of the invention is to present a method of producing a humidity sensor with fast response (0.12-0.28 seconds) and/or recovery times (0.40-2.80 seconds).

The structural and characteristic features of the invention and all its advantages will be understood more clearly by means of the figures given below and the detailed explanation written by making references to these figures. Therefore, the evaluation should be made by taking these figures and detailed explanation into consideration.

### BRIEF DESCRIPTION OF THE DRAWINGS

In order to for the embodiment of the present invention and its advantages with additional elements to be understood in the best way, it should be evaluated together with the figures described below.

FIG. 1. The representative view of the spray accumulation system of the humidity sensor production method that is the subject of the invention.

FIG. 2. Representative view of the resulting NiOx humidity sensor.

FIG. 3a. SEM image of the obtained NiOx humidity sensor at ×90 magnification.

FIG. 3b. SEM image of the obtained NiOx humidity sensor at ×500 magnification.

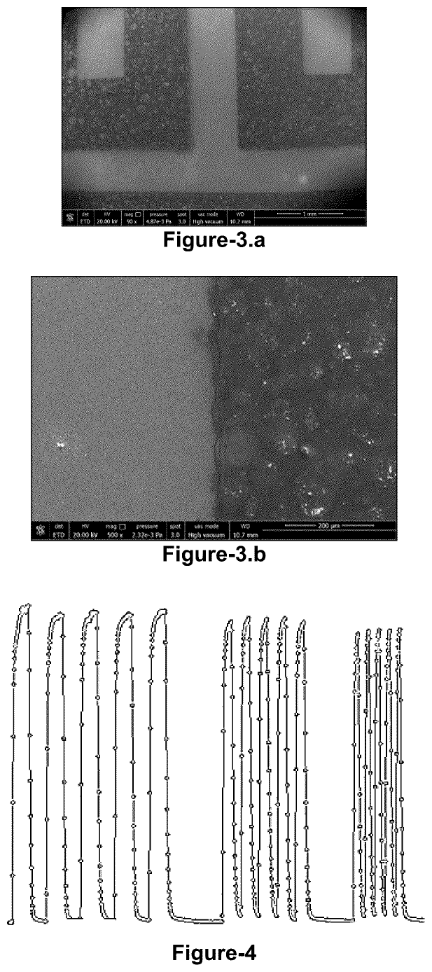

FIG. 4. The dynamic response graph of the NiOx humidity sensor obtained is at frequencies of 0.5 Hz, 1 Hz and 2 Hz from left to right, respectively, while the ambient relative humidity changes between 32% and 75%.
FIGS. 3a and 3b are presented in colour and filled because they are magnified SEM microscope graphics.
Other graphics in the description are presented in the detailed description section due to the quantitative values around them.

### REFERENCE NUMBERS

1. Humidity Sensor
2. Solution
3. Spray
4. Air Compressor
5. Pressure Gauge
6. Thermocouple
7. Heater
8. Base
9. Electrode
d. Spraying Distance

### DETAILED DESCRIPTION OF THE INVENTION

In this detailed description, the production method of the humidity sensor (1) which is the subject of the invention, and its preferred embodiments are explained only for the purpose of a better understanding of the subject and in a way that does not create any limiting effect.

The humidity sensor (1) production method that enables the production of a NiOx (Nitric Oxide) based humidity sensor (1) formed by the accumulation method for use in the production of MOS (Metal Oxide Semiconductor) sensors, which is the subject of the invention, comprises the process steps of:
preparing nickel acetate tetra dihydrate and deionised pure water solution (2),
forming interdigitated electrodes (9) by accumulating silver on the glass sensor base (8) by the thermal evaporator,
spraying the prepared solution (2) on the silver electrodes (9) accumulated on the glass sensor base (8) on the heater (7), and
accumulating the sprayed solution (2) on the silver electrodes (9) accumulated on the sensor base under the effect of the heater (7) and forming the humidity sensor (1).

In a preferred embodiment of the invention, after the process step of “accumulating the sprayed solution (2) on the silver electrodes (9) accumulated on the sensor base under the effect of the heater (7) and forming the humidity sensor (1)”, there is also a process step of leaving the solution (2) accumulated on the silver electrodes (9) accumulated on the sensor base to cool down at room temperature.

In a preferred embodiment of the invention, before the process step of “spraying the prepared solution (2) on the silver electrodes (9) accumulated on the glass sensor base (8) on the heater (7)”, there also is a process step of operating the sprayed accumulation system idle for 10 minutes without adding the solution (2) and without placing the base (8) on the heater (7) in order to fix the temperature and air pressure.

In a preferred embodiment of the invention, there is a process step of rapidly annealing the humidity sensor (1) on a heated table at 450° C. for 5-10 minutes at the end of production.

A preferred embodiment of the invention comprises the process step of controlling the temperature values of the glass base (8) by a thermocouple (6) to ensure that they remain within a predefined range throughout all process steps.

### A Preferred Embodiment of the Invention Works as Follows

a solution of nickel acetate tetra dihydrate and deionised pure water (2) is prepared,

Solution (2) is prepared in low molarity under atmospheric and room conditions using “nickel acetate tetra dihydrate” source material and deionised pure water. The solution is prepared with 0.05 M molarity using magnetic stirrer at room temperature at 300 RPM for 30 minutes.
silver is accumulated onto the glass sensor base (8) by the thermal evaporator and interdigitated electrodes (9) are formed,

Interdigitated electrodes (9) with a thickness of 125-150 nm are manufactured on a 1×2.5 cm glass sensor base (8) with dimensions in the centimetre range, thickness and spacing in the micron range, and patterned with high purity (99.999%) silver. The electrodes (9) are formed by the physical vapour accumulation method, using tungsten crucibles with a current of 60-70 A passing through them under a 10⁻⁵ Torr vacuum, without heating the glass sensor bases (8).
The spray accumulation system is run idle for 10 minutes without adding solution (2) to stabilise the temperature at 300° C. and the air pressure at 40 PSI,

Thus, the system is stabilised.

Before the spraying process, the prepared solution (2) is dropped into the spray (3) chamber to be sprayed at a flow rate of 0.03 ml/s using gravity.
The prepared solution (2) is sprayed onto the silver electrodes (9) accumulated on the glass sensor base (8) on the heater (7),

A simple spray accumulation system is created using an injection needle tip placed in the spray (airbrush) (3) solution chamber for the spraying process, a thermocouple (6), a magnetic stirrer/heater (7) and an air compressor (4). The air pressure is fixed, and the solution (2) flow rate is kept under control. In this way, the air flow balance point is reached and a smooth and homogeneous flow that can be sprayed stably is obtained. Before starting the spraying process, the prepared glass base (8) (glass coated with interdigitated Ag electrodes (9)) is placed in the centre on the heater (7) with the interdigitated Ag electrode (9) surface to be coated facing the sprayer.

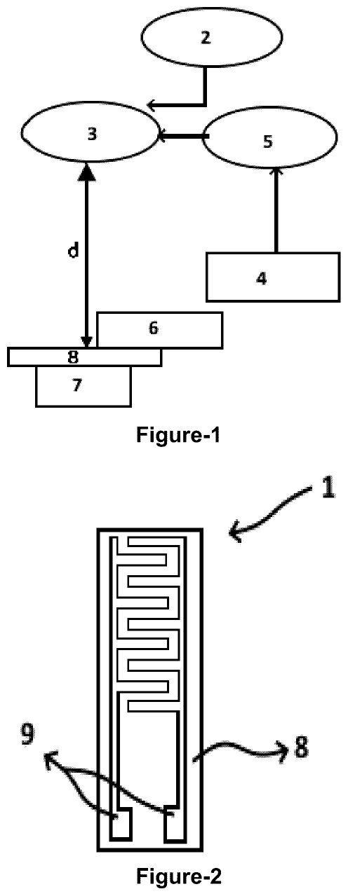

In this system shown in FIG. 1, the NiOx starting solution (3) prepared above was sprayed onto Ag interdigitated (patterned) silver electrodes (9) by reducing the pressure to 40 PSI with the atmospheric air compressor (4) at 100 PSI pressure and sprayed from the spray (3) nozzle.
The sprayed solution (2) accumulates on the silver electrodes (9) accumulated on the sensor base under the effect of the heater (7) and forms the humidity sensor (1),

The solution (2) is sprayed onto the silver electrodes (9) accumulated on the glass sensor base (8) on the heater (7) by the air compressor (4) at 40 PSI pressure. The spraying process is carried out from a certain spraying distance (d) with an injection needle attached to the solution chamber of the spray (3) into which the solution (2) is placed, and the production is considered accepted when the solution (2) reaches the tip of the needle. The coating process is completed by spraying approximately 15-20 ml of solution (2).
the solution (2) accumulated on the silver electrodes (9) accumulated on the glass sensor base (8) is left to cool at room temperature,

After the accumulation process is completed, the sensor is left to cool at room temperature with the heater (7) turned off and air flow provided to prevent any increase in temperature.
placing the humidity sensor (1) on the heater (7) preheated to 450° C. and annealing rapidly for 5-10 minutes to provide a humidity sensor (1) that is not sufficiently saturated with oxygen but whose crystal structures have improved,
a thermocouple (6) controls the temperature values of the glass sensor base (8) within a predefined range throughout all process steps.

A thermocouple (6) controls the temperature values of the glass sensor base (8) within a predefined range throughout all process steps. The temperature readings of the actual base are taken and monitored before, during and after accumulation. The reading of the thermocouple (6) is measured and checked, especially when the system equilibrium is reached and also during accumulation. The thermocouple (6) is fixed with a heat-resistant Kapton tape, just touching the edge of the glass sensor base (8).

### Characterisation of Sensors:

The humidity responses of the sensors were made at different gas flow rates and humidity densities manipulated with computer-controlled Alicat brand gas flow control units. Electrical measurements were also monitored with a computer-controlled Keithley 2611 brand source meter system.

The final product was checked for repeatability stability and response/recovery time and the sensor response results are shown in graph 1.

The response time is the time it takes for the sensor to measure the amount of moisture when exposed to moisture, while the recovery time is the time it takes for the sensor to become suitable for a new measurement after the measurement. The response and recovery times are interrelated parameters and in said invention, one of these parameters can be waived and the other can be changed. The “x” in the NiOx expression defines the oxygenation level of the compound and the response time and recovery time can be adjusted by adjusting how many oxygen bonds will be established/vacated. A decrease in the oxygenation rate will mean a decrease in the response time, i.e. the sensor will speed up, and in this case, the recovery time will be extended according to the number of oxygen bonds established/vacated, i.e. a late recovery will occur. Since the total number of oxygen bonds is limited, both of these times naturally have a limit. The oxygenation rate is directly related to the duration and temperature of the rapid annealing process. Therefore, if the duration and degree of annealing are changed, the response and recovery times can also be changed.

The preferred annealing temperature of 450 degrees and 5-10 minutes is given for optimum response and recovery time in the invention. The optimum values of response and recovery times are (0.28 seconds) for response and (0.40 seconds) for recovery. Depending on the use of the sensor to be produced, the annealing temperature and/or time can be changed to go beyond these values.

The detection range of relative humidity was verified with the wet finger test, and it is known that the relative humidity of the wet finger is high, also the response was examined as a function of the fingertip distance and is shown in graph 2.

The sensors' ability to operate at voltages much lower than 0.1 V is shown in graph 3 and one of the biggest technical problems of humidity sensors (1) has been solved. It is seen from the small inner graph in graph 3 that a response can be easily obtained at 0.1 V and below.

The XRD analyses of the NiOx coatings forming the sensors and the “cubic crystal structure NiO” match belonging to the COD library (COD #9008693) are given in graph 4.

In the field of metal oxide semiconductor (MOS) sensors, a NiOx-based humidity sensor (1) that is cost-effective, fast-response, high-sensitivity, has a wide relative humidity sensing range, is stable, has a long life, can be quickly recovered, and can operate at room temperature and with low voltage by means of the silver (Ag) interdigitated electrode (9) contacts using the accumulation method has been presented with this method. A fast-response, fast-recovery, stable humidity sensor (1) that can operate at room temperature and low voltages in a wide range of relative humidity % with NiOx material, using the spray (airbrush) (3) technique, which is a cost-effective thin film production method, and using silver metal that is much cheaper and more easily available/coated than other expensive metal electrodes (9). Said sensor can be used in all industrial branches that include the use of humidity and gas sensors, in wearable technologies and in the healthcare sector (as a breath detection/evaluation sensor) for applications in healthy/unhealthy or disease diagnosis with breath patterns.

1. A humidity sensor production method that enables the production of a NiOx (Nitric Oxide) based humidity sensor formed by the accumulation method for use in the production of MOS (Metal Oxide Semiconductor) sensors, comprising the process steps of:
preparing nickel acetate tetra dihydrate and deionised pure water solution,
forming interdigitated electrodes by accumulating silver on the glass sensor base by the thermal evaporator,
spraying the prepared solution on the silver electrodes accumulated on the glass sensor base on the heater, and
accumulating the sprayed solution on the silver electrodes accumulated on the sensor base under the effect of the heater and forming the humidity sensor.

2. The humidity sensor production method according to claim 1 further comprises the process step of leaving the solution accumulated on the silver electrodes accumulated on the sensor base to cool down at room temperature after the process step of “accumulating the sprayed solution on the silver electrodes accumulated on the sensor base under the effect of the heater and forming the humidity sensor.

3. The humidity sensor production method according to claim 1 further comprises the process step of operating the spray accumulation system idle for 10 minutes without adding solution to stabilise the temperature at 300° C. and the air pressure at 40 PSI before the process step of spraying the prepared solution on the silver electrodes accumulated on the glass sensor base on the heater.

4. The humidity sensor production method according to claim 1 further comprises the process step of placing the humidity sensor on the heater preheated to 450° C. and annealing rapidly for 5-10 minutes to provide a humidity sensor that is not sufficiently saturated with oxygen but whose crystal structures have improved after the process steps of accumulating the sprayed solution on the silver electrodes accumulated on the sensor base under the effect of the heater and forming the humidity sensor or leaving the solution accumulated on the silver electrodes accumulated on the sensor base to cool down at room temperature.

5. The humidity sensor production method according to claim 1 further comprises the process step of preparing 0.05 M molarity nickel acetate tetra dihydrate and deionized pure water solution using a magnetic stirrer at room temperature at 300 RPM for 30 minutes in the process step preparation of nickel acetate tetra dihydrate and deionized pure water solution.

6. The humidity sensor production method according to claim 1 further comprises the process step of forming interdigitated electrodes by accumulating silver on the glass sensor base by a thermal evaporator without heating the glass sensor base using tungsten crucibles with a current of 60-70 A under a 10⁻⁵ Torr vacuum using the physical vapour accumulation method in the process step of forming interdigitated electrodes by accumulating silver on the glass sensor base by the thermal evaporator.

7. The humidity sensor production method according to claim 1 further comprising the process step of dropping the prepared solution into the sprayer to be sprayed at a flow rate of 0.03 ml/s before the process step of spraying the prepared solution on the silver electrodes accumulated on the glass sensor base on the heater.

8. The humidity sensor production method according to claim 1 further comprising the process step of spraying the prepared solution onto the silver electrodes deposited on the glass sensor base on the heater at 40 PSI pressure with the air compressor in the process step of spraying the prepared solution on the silver electrodes accumulated on the glass sensor base on the heater.

9. The humidity sensor production method according to claim 1 further comprising the process step of controlling the temperature values of the glass sensor base by a thermocouple to ensure that they remain within a predefined range, throughout all process steps.
